(19) United States
(12) Patent Application Publication
(10) Pub. No.: US 2014/0303729 A1
(43) Pub. Date: Oct. 9, 2014

(54) INTERFERENCE WEDGE FOR SECURING AN ANTERIOR CRUCIATE LIGAMENT GRAFT IN A BONE TUNNEL

(71) Applicant: James M. LEE, Orange, NJ (US)

(72) Inventor: James M. LEE, Orange, NJ (US)

(21) Appl. No.: 13/859,092

(22) Filed: Apr. 9, 2013

(51) Int. Cl. A61F 2/08

(52) U.S. Cl. CPC A61F 2/0811; A61F 2002/0858
USPC 623/13.12

(57) ABSTRACT

A wedge is used to engage on one side with a femoral tunnel or a tibial tunnel and on the opposite side engage with a bone plug. This is accomplished by forming a wedge being substantially semi-circular on one side and flat on the opposite side.

### FIELD OF THE INVENTION

The present invention relates to the use of an interference wedge to secure a bone plug of a tendonous graft in place in a bone tunnel.

### BACKGROUND OF THE INVENTION

During the repair of an anterior cruciate ligament (ACL), a tendonous graft is formed by a tendon between two pieces of bone. The bone pieces are fit into a tibial tunnel and a femoral tunnel so as to interconnect the tibia and femur.

Often times a screw is used to secure the bone piece in the appropriate tunnel. A problem encountered is that the screw engages and at least partially tears the tendon interconnecting the bone pieces. This weakens or destroys the interconnecting tendon.

### SUMMARY OF THE INVENTION

Accordingly, it is an object of the present invention to avoid the drawbacks of the prior art procedures.

This object is accomplished by the use of a wedge which is used to engage on one side with a femoral tunnel or a tibial tunnel and on the opposite side engage with a bone plug. This is accomplished by forming a wedge being substantially semi-circular on one side and flat on the opposite side.

The semi-circular side is made of trabecular metal having a series of roughened protrusions varying in height from 0.25 to 2.5 mm, and preferably 1.25 mm in height. The roughened semi-circular surface securely fixes the bone plug in place. Further, the roughened surface causes the expected bone regeneration of approximately 2 mm to attach to the wedge and securely fasten the wedge in place in the bone tunnel.

The substantially smooth underside of the wedge contacts the bone plug and positions the plug in place. The smooth surface of the wedge avoids the possibility of chewing up the graft as is experienced by the use of an interference screw.

At a trailing edge of the wedge is a hook that engages the bone plug or is used to loop the bone tendon. This helps guide the bone plug in place in the femoral tunnel.

A hole in a center of the proximal end of the wedge is used for engagement by a long slender driver. The driver will engage the wedge in a slot in the bottom of the hole to rotate the wedge into position in the bone tunnel by rotation of the driver. The driver is also used to push the wedge to its final resting position. The driver is then backed away from the wedge and removed.

The positioning of the tendonous graft may be accomplished by passing the bone plug at one end of the graft through the tibial tunnel until reaching the femoral tunnel. The wedge is then engaged with the bone plug in the femoral tunnel. The plug is then moved into position in the femoral tunnel by the slender driver until reaching its final position as determined by the surgeon.

Accordingly, it is another object of the present invention to secure a tendonous graft in place between a femur and a tibia in a respective bone tunnel.

It is another object of the present invention to secure a tendonous graft in place between a femur and a tibia in a respective bone tunnel by the use of a wedge engaging on one side with the bone tunnel and on the opposite side with the bone plug.

It is still yet another object of the present invention to secure a tendonous graft in place between a femur and a tibia in a respective bone tunnel by the use of a wedge engaging on one side with the bone tunnel and on the opposite side with the bone plug with the surface engaging the bone tunnel being curved and covered with trabecular metal and the side engaging the bone plug being substantially smooth.

It is another object of the present invention to secure a tendonous graft in place between a femur and a tibia in a respective bone tunnel by the use of a wedge engaging on one side with the bone tunnel and on the opposite side with the bone plug with the surface engaging the bone tunnel being curved and covered with trabecular metal and the side engaging the bone plug being substantially smooth and a hook at a proximal end of the wedge engaging at least one of the bone plug and the tendon of the graft to assist in the positioning of the tendonous graft.

These and other objects of the invention, as well as many of the intended advantages thereof, will become more readily apparent when reference is made to the following description taken in conjunction with the accompanying drawings.

### BRIEF DESCRIPTION OF THE DRAWINGS

The following drawings illustrate examples of various components of the invention disclosed herein, and are for illustrative purposes only. Other embodiments that are substantially similar can use other components that have a different appearance.

FIG. 1 is a perspective view of the wedge of the present invention.

FIG. 2 is a side view of the wedge shown engaging a bone plug and hooking a tendon of a tendonous graft.

FIG. 3 shows the bottom surface of the wedge.

FIG. 4 illustrates the positioning of a tendonous graft in a femoral tunnel.

FIG. 5 illustrates the passage of the tendonous graft through a tibial tunnel for engagement of one end of the tendonous graft in the femoral tunnel.

FIG. 6 illustrates the positioning of a driver to engage a hole in the base of the wedge for twisting and moving of the wedge into the femoral tunnel while engaging a bone plug.

### DETAILED DESCRIPTION OF THE PREFERRED EMBODIMENTS

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents which operate in a similar manner to accomplish a similar purpose.

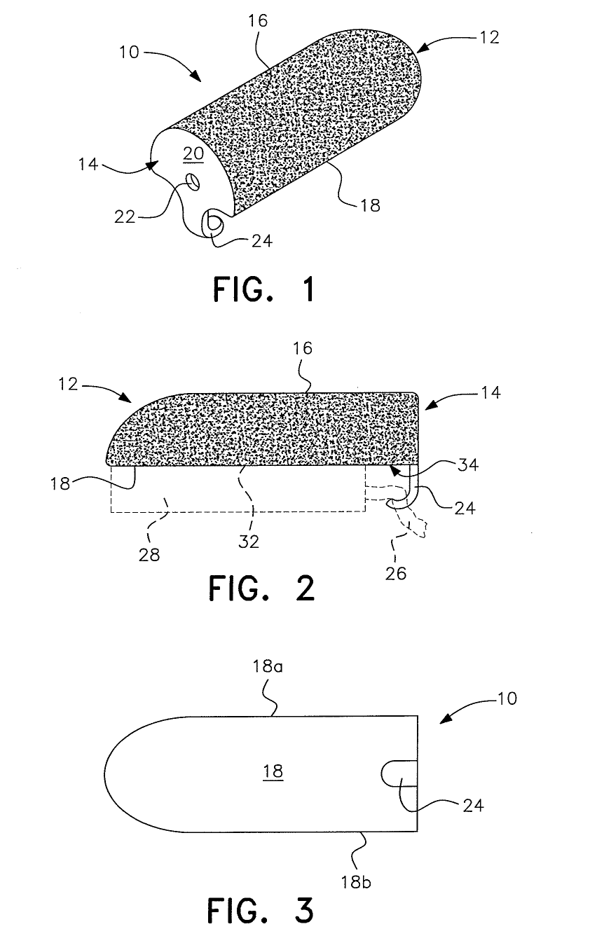

With reference to the drawings, in general, and to FIGS. 1 through 3, in particular, a wedge embodying the teachings of the subject invention is generally designated as 10. With reference to its orientation in FIG. 1, the wedge includes a distal end 12 and a proximal end 14. The wedge is made of a biocompatible metal material.

The upper surface 16 of the wedge is curved, semi-circular in shape having a radius of approximately 4.5 mm. Towards the distal end and the lateral side edges, the upper surface 16 tapers along a curved line to the lower surface 18. The lower surface is substantially flat. A distance between opposed edges 18a and 18b of the lower surface 18 is approximately 9 mm.

The upper surface 16 is coated with trabecular metal substantially across its entire surface. The trabecular metal approaches the size and distribution of various shaped spicules of bone found in cancellous tissue. This type of surface fosters the growth of bone into the surface of the wedge for a secure anchoring of the wedge in a bone tunnel.

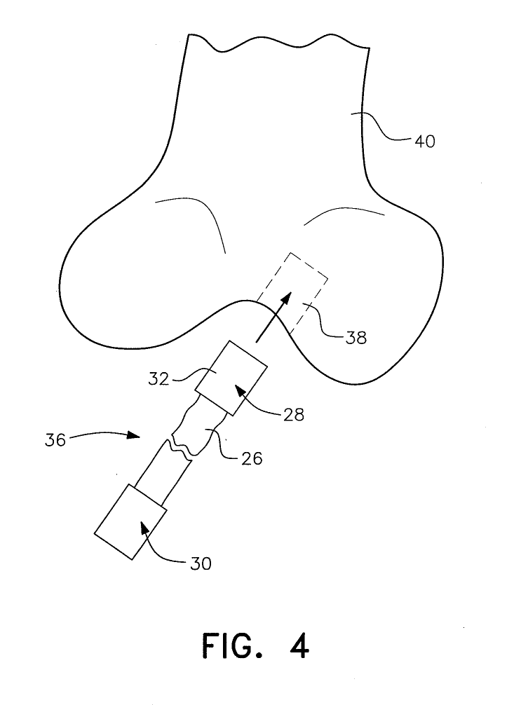

At the proximal end 14 is a flat base surface 20 extending substantially perpendicular to the lower surface 18. The base surface 20 includes a hole 22 which extends approximately 3 to 5 mm into the wedge. A slot may be located at the base of the hole 22. Extending from the base surface 20 towards the bottom surface 18 is a hook 24. The hook 24, as shown in FIG. 2, is used to engage a tendon 26 of a tendonous graft formed by the tendon 26 extending between two bone plugs 28, 30, as shown in FIG. 4.

As shown in FIG. 2, the lower surface 18 of the wedge 10 engages with the upper surface 32 of the bone plug 28. In the space 34, between the end of the bone plug 28 and the base surface 20 of the wedge, may also be located some trabecular metal, located only adjacent to the proximal end 14 of the wedge.

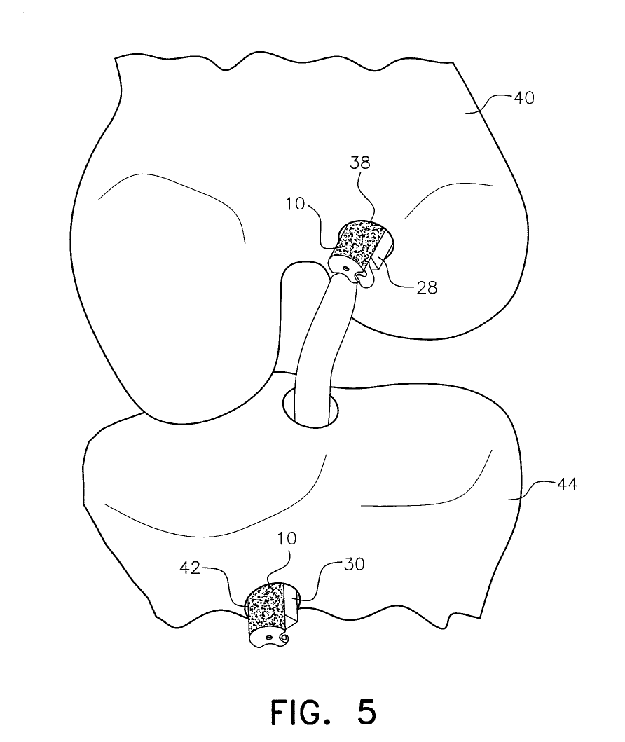

As shown in FIG. 4, the tendonous graft 36 includes two bone plugs 28, 30 interconnected by a tendon 26. It is the object of the invention to secure the bone plug 28 in a femoral tunnel 38 of femur 40. The opposite bone plug 30 is to be secured in a tibial tunnel 42 of a tibia 44, as shown in FIG. 5.

In FIG. 5, a wedge 10 is positioned on top of bone plug 28 partially inserted into femoral tunnel 38. The bone plug 28 has been pushed through tibial tunnel 42 to reach femoral tunnel 38 without additional surgical incisions. A wedge 10 may be positioned on top of bone plug 30 while passing through tibial tunnel 42. The hook 24 may engage a trailing edge of the bone plug 28 to help move the bone plug 28. By forming the tibial tunnel 42 of a greater diameter than the femoral tunnel 38, it is possible to push the bone plug 28 and wedge 10 through the tibial tunnel until reaching the femoral tunnel. Then, by use of a slightly larger wedge, the bone plug 30 may be secured in place in the tibial tunnel by the slightly enlarged size wedge 10.

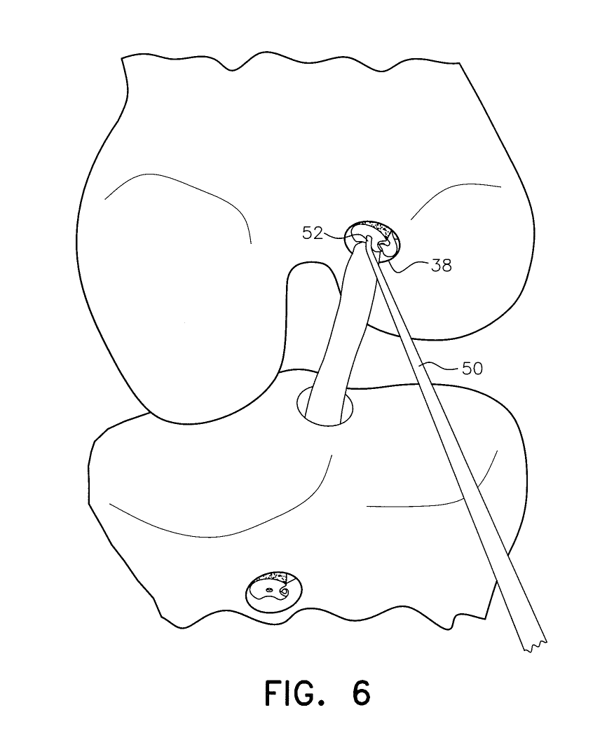

As shown in FIG. 6, a driver 50 having a narrowed tip 52 engages in the hole 22 of the base surface 20 of the wedge. The tip 52 may engage the slot in the bottom of the hole 22 so as to rotate the wedge to a desired position and then, by driving the driver, the wedge and the bone plug 28 may be secured in position within the femoral tunnel 38.

The foregoing description should be considered as illustrative only of the principles of the invention. Since numerous modifications and changes will readily occur to those skilled in the art, it is not desired to limit the invention to the exact construction and operation shown and described, and, accordingly, all suitable modifications and equivalents may be resorted to, falling within the scope of the invention.

I claim:

1. An interference wedge for securing a bone plug in a bone tunnel, said interference wedge comprising
a body having a distal end, a proximal end, an upper surface and a lower surface,
said upper surface being curved between lateral side edges and towards the distal end, the upper surface being covered with trabecular metal,
the lower surface being substantially flat.

2. The interference wedge according to claim 1, wherein a base surface at said proximal end extends substantially perpendicular to said lower surface between said upper surface and said lower surface.

3. The interference wedge according to claim 2, wherein said base surface includes a hole for engagement by a driver.

4. The interference wedge according to claim 3, wherein a slot is located at a bottom of the hole.

5. The interference wedge according to claim 2, wherein a hook extends from said base surface beyond said lower surface.

6. The interference wedge according to claim 5, wherein said hook extends from said proximal end towards said distal end.

7. The interference wedge according to claim 1, wherein said trabecular metal extends from said upper surface to a height of approximately 1.25 mm.

8. The interference wedge according to claim 1, wherein only a portion of said lower surface at said proximal end includes trabecular metal.

9. The interference wedge according to claim 1, wherein the body is made of biocompatible material.

10. The interference wedge according to claim 9, wherein the biocompatible material is metal.
